(19) United States
(12) Patent Application Publication
(10) Pub. No.: US 2005/0124953 A1
(43) Pub. Date: Jun. 9, 2005

(54) Absorbent article with segmented and separated absorbent structure

(75) Inventors: Garry Woltman, Greenville, WI (US); Katherine C. Wheeler, Menasha, WI (US); Barb O. Sauer, Fremont, WI (US)

Correspondence Address: DORITY & MANNING, P.A., POST OFFICE BOX 1449, GREENVILLE, SC, 29602-1449, US

(73) Assignee: Kimberly-Clark Worldwide, Inc.

(21) Appl. No.: 10/727,892

(22) Filed: Dec. 4, 2003

(51) Int. Cl. A61F 13/15; A61F 13/20

(52) U.S. Cl. 604/385.01

(57) ABSTRACT

The present invention discloses and claims an absorbent article having a liquid permeable cover, a generally liquid impermeable baffle, and segmented absorbent pads that are transversely separated by a flexible zone. The flexible zone is minimally absorbent and preferentially deforms before the segmented absorbent pads. Lateral force on the absorbent article compresses the flexible zone and reduces the transverse width of the absorbent article.

### BACKGROUND OF THE INVENTION

A typical absorbent article includes a liquid permeable top sheet, a liquid impermeable back sheet, and an absorbent core or structure disposed between the top sheet and back sheet. A user wears the absorbent article in the crotch area of an undergarment. Exudates, such as urine and menses, pass through the top sheet for absorption by the absorbent core, and the back sheet prevents any exudates from penetrating the absorbent article and reaching the undergarment. The user generally wears the absorbent article a single time and discards the article after a relatively short period of use.

The length, width, and thickness of the absorbent article depend on the particular application and intended user. Ideally, the absorbent article should fully cover the user's genital area, possess maximum fluid capacity, remain visually discrete from the outside of the undergarment, and minimize discomfort to the user. In practice, fully covering the user's genital area and possessing sufficient fluid capacity increases the length, width, and/or thickness of the absorbent article, resulting in an article that is less visually discrete from the outside of the undergarment and/or less comfortable to the user.

Various designs of absorbent articles exist to optimize the competing design considerations of coverage, capacity, visual discretion, and comfort. For example, U.S. Pat. No. 6,160,197 discloses an absorbent article having an absorbent core positioned between a fluid-permeable cover and a fluid-impermeable baffle. The absorbent core further includes longitudinal flexure axes that permit the absorbent core to fold into a “W” shape when subjected to lateral compressive forces, such as the user's legs. In this manner, the absorbent core can be made wider to cover a larger area and possess additional fluid capacity, yet the absorbent core may collapse to a “W” shape when compressed to reduce the width and promote comfort.

U.S. Pat. No. 5,514,104 discloses another attempt to improve the coverage and capacity of the absorbent core without sacrificing visual discretion or comfort. As disclosed in U.S. Pat. No. 5,514,104, the posterior and/or anterior end of the absorbent article includes a notch. The notch permits that end of the absorbent article to conform to and move with the user's body, improving the article's visual discretion and improving the user's comfort.

Other geometries exist to improve the balance between coverage, capacity, visual discretion, and comfort by varying the geometry of the absorbent article and/or absorbent core, and the present invention provides another such geometry.

### SUMMARY OF THE INVENTION

Objects and advantages of the invention are set forth below in the following description, or may be obvious from the description, or may be learned through practice of the invention.

In one embodiment of the invention, a feminine care absorbent article includes a liquid permeable top sheet, a generally liquid impermeable back sheet peripherally joined to the top sheet, and an absorbent core between the top sheet and back sheet. The absorbent core includes a first longitudinal segment and a second longitudinal segment transversely separated from the first longitudinal segment. The absorbent article further includes a flexible zone between the first and second longitudinal segments, and the flexible zone has a reduced absorbency compared to the absorbent core. In a particular embodiment, the feminine care absorbent article may also include a transverse segment proximate to either or both ends of the first and second longitudinal segments.

In another embodiment, an absorbent article includes a liquid permeable top sheet, a generally liquid impermeable back sheet peripherally joined to the top sheet, a plurality of longitudinal absorbent segments between the top sheet and back sheet, and a flexible zone between the longitudinal absorbent segments. The flexible zone has a reduced thickness compared to the longitudinal absorbent segments. In a particular embodiment, the absorbent article may also include a transverse segment proximate to either or both ends of the longitudinal segments.

An alternate embodiment of the present invention includes a liquid permeable cover, a generally liquid impermeable baffle peripherally joined to the cover, and an absorbent core between the cover and baffle. The absorbent core has a transverse axis with a stepped absorbent capacity and a stepped flexibility. The absorbent core may also have a longitudinal axis with a substantially constant absorbent capacity and a substantially constant flexibility.

In yet another embodiment, a feminine care absorbent article includes a liquid permeable cover, a generally liquid impermeable baffle peripherally joined to the cover, and an absorbent core between the cover and baffle. The absorbent core includes a first longitudinal segment and a second longitudinal segment transversely separated from the first longitudinal segment. The absorbent core has an extended position in which the first and second longitudinal segments are transversely separated by approximately 10 millimeters. The absorbent core also has a compressed position in which the first and second longitudinal segments are substantially together.

Those of ordinary skill in the art will better appreciate the features and aspects of such embodiments, and others, upon review of the specification.

### BRIEF DESCRIPTION OF THE DRAWINGS

A full and enabling disclosure of the present invention, including the best mode thereof to one skilled in the art, is set forth more particularly in the remainder of the specification, including reference to the accompanying figures, in which:

FIG. 1 is a perspective view with partial cut-away of an embodiment of the present invention;

FIG. 2A is a perspective view with partial cut-away of an embodiment of the present invention without any lateral compression;

FIG. 2B is a perspective view with partial cut-away of an embodiment of the present invention under lateral compression; and

FIGS. 3, 4, and 5 are perspective views with partial cut-away of alternate embodiments of the present invention.

### DETAILED DESCRIPTION OF PREFERRED EMBODIMENTS

Reference will now be made in detail to present embodiments of the invention, one or more examples of which are illustrated in the accompanying drawings. Each example is provided by way of explanation of the invention, not limitation of the invention. In fact, it will be apparent to those skilled in the art that modifications and variations can be made in the present invention without departing from the scope or spirit thereof. For instance, features illustrated or described as part of one embodiment may be used on another embodiment to yield a still further embodiment. Thus, it is intended that the present invention covers these and other such modifications and variations as come within the scope of the appended claims and their equivalents.

The present invention relates generally to an absorbent article 10 for intercepting and retaining body fluids or exudates. The accompanying figures depict the absorbent article 10 as a sanitary napkin for feminine hygiene having a conventional elongated oval shape; however, the absorbent article 10 may also be a panty liner, shield, diaper, training pants, adult incontinent garment, or any other disposable absorbent article known in the art. Moreover, the absorbent article 10 may have other shapes, such as hourglass or rectangular, and varying sizes and thickness, depending on the particular application.

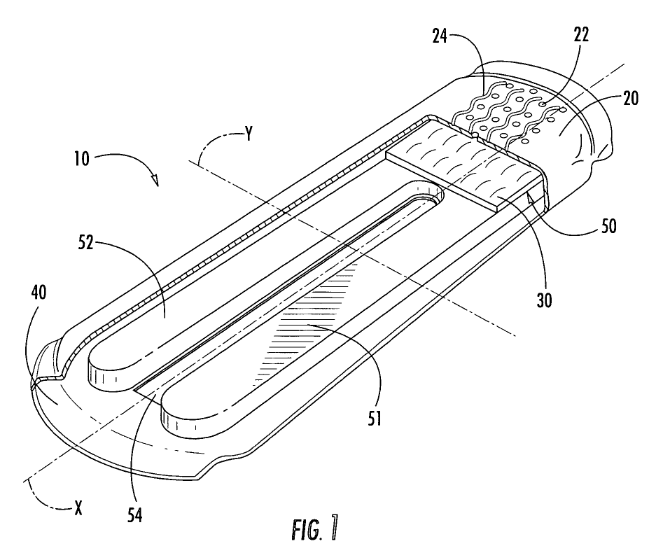

Referring to FIG. 1, the absorbent article 10 generally includes a top sheet or cover 20, an optional transfer layer 30, a back sheet or baffle 40, and an absorbent core 50.

The top sheet or cover 20 provides the absorbent article 10 with a liquid permeable surface that contacts the user's skin. The cover 20 should provide a comfortable, conforming interface with the user's skin by being flexible, compliant, and non-irritating to the skin. The cover 20 should also transfer fluids quickly and remain dry and clean during use. In addition to being liquid permeable, the cover 20 may also include apertures 22 for freely passing exudates with minimal absorption. The cover 20 may be coated with a surfactant to further enhance permeability to the absorbent core 50 and reduce absorption by the cover 20. The cover 20 may also include embossed channels 24 to create an aesthetically pleasing surface and further disperse exudates passing through the cover 20.

The cover 20 may be any woven or non-woven material which passes body fluids yet remains comfortable to the user. Examples of suitable cover materials include rayon, bonded carded webs of polyester, polypropylene, polyethylene, nylon, or other heat-bondable fibers, polyolefins, copolymers of polypropylene and polyethylene, linear low-density polyethylene, and aliphatic esters such as polylactic acid. A specific example of a suitable cover material is a bonded carded web made of polypropylene and polyethylene such as that used as cover stock for KOTEX® panty liners and obtainable from Sandler Corporation, Germany.

The transfer layer 30 provides an optional layer between the cover 20 and the absorbent core 50. When present, the transfer layer 30 wicks fluid passing through the cover 20 and disperses the fluid to the absorbent core 50. The transfer layer 30 may comprise a fibrous blend of polyester, rayon, and a polymeric fiber such as that marketed by C. Itoh & Co. under the trademark CHISSO. For example, the transfer layer 30 may be 50 percent polyester fibers having a length of approximately three to six millimeters, approximately 30 percent straight rayon fibers having a length of approximately five to ten millimeters, and the balance CHISSO having a length of approximately five to ten millimeters.

The back sheet or baffle 40 may peripherally join to the cover 20 and provide the absorbent article 10 with a liquid impermeable and possibly vapor permeable surface that prevents exudates from completely penetrating the absorbent article 10 and soiling the user's undergarment. Ideally, the baffle 40 is soft, flexible, quiet, breathable, and may include some absorbent capacity on the side facing the absorbent core 50. The garment-facing side of the baffle 40 may include adhesive strips or other suitable fastening device (not shown) for adhering the absorbent article 10 to the user's undergarment. The baffle 40 may be any suitable material known in the art, such as embossed and non-embossed thermoplastic films, laminated tissue, and polyethylene films. In one embodiment, the baffle 40 includes a non-woven material laminated to a microporous film.

The absorbent core 50 provides the operative material for collecting and retaining body fluids or exudates while remaining light and dry feeling during use. The absorbent core 50 should be soft, not stiff, and should retain its shape, even when wet. The absorbent core 50 resides between the cover 20 and baffle 40 and may be attached to either or both layers to hold the absorbent core 50 in place and protect the absorbent core 50 from abrasion. The absorbent core 50 is typically approximately 2-10 millimeters thick and is oriented about a longitudinal axis X and a transverse axis Y.

The absorbent core 50 may be any structure or combination of components which are generally compressible, conformable, non-irritating to the user's skin, and capable of absorbing and retaining bodily fluids. For example, the absorbent core 50 may include an absorbent web of cellulose fibers, such as wood pulp fibers, other natural fibers, synthetic fibers, woven or non-woven sheets, scrim netting or other stabilizing structures, superabsorbent materials, binder materials, surfactants, selected hydrophobic and hydrophilic materials, pigments, lotions, and odor control agents, as well as combinations thereof. In one embodiment, the absorbent core 50 is a matrix of cellulose fluff coated with superabsorbent hydrogel-forming particles. The absorbent core 50 may be formed using various methods and techniques known in the art, such as dry-forming, air forming, wet-forming, and foam-forming, as well as combinations thereof.

Superabsorbent materials are well known in the art and may be selected from natural, synthetic, and modified natural polymers and materials. The absorbent core 50 generally includes superabsorbent material, with the superabsorbent material ranging from about 0-90 percent by weight of the absorbent core 50, depending on the application and desired absorbency. For example, the total absorbency may be about 200-900 grams of 0.9% by weight saline for infant care products; whereas, the total absorbency for adult care products may be about 400-2000 grams of 0.9% by weight saline. For feminine care products, the total absorbency may be within the range of about 7-50 grams of menstrual fluid.

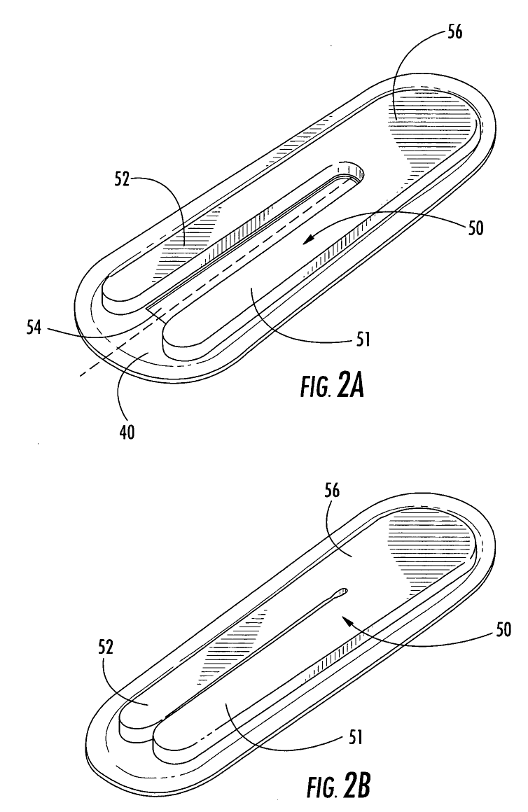

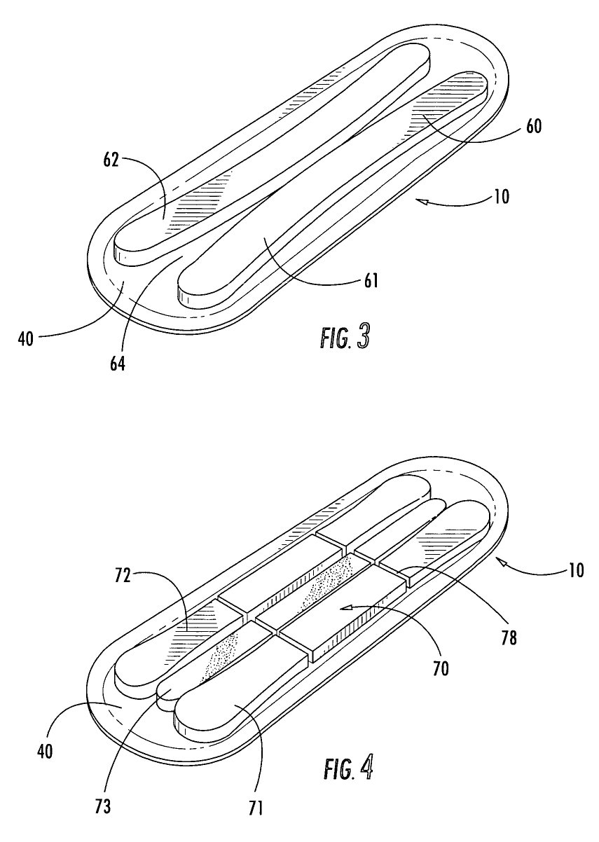

Referring to FIGS. 1, 2A, and 2B, in one embodiment the absorbent core 50 generally includes two longitudinal absorbent segments 51, 52, and a flexible zone 54 separates the two longitudinal absorbent segments 51, 52. In this configuration, the longitudinal absorbent segments 51, 52 extend substantially parallel to the longitudinal axis X and occupy the outer edge of the absorbent article 10. The longitudinal absorbent segments 51, 52 may vary in width and separation along the longitudinal axis X and generally have a combined width of absorbent material of approximately 10-50 millimeters. In particular embodiments, the longitudinal absorbent segments 51, 52 may be thicker and/or more absorbent adjacent to the longitudinal axis X and/or at the transverse axis Y, with the thickness and/or absorbency decreasing as the longitudinal absorbent segments 51, 52 extend away from the longitudinal axis X and/or the transverse axis Y.

In the embodiment shown in FIGS. 1, 2A, and 2B, the absorbent core 50 also includes a transverse absorbent segment 56 generally parallel to the transverse axis Y. The transverse absorbent segment 56 may occupy either or both ends of the absorbent article 10 and may be integral to the longitudinal absorbent segments 51, 52, as shown, or separate from them. The transverse absorbent segment 56 generally has an absorbent width approximately equal to the sum of the widths of the longitudinal absorbent segments 51, 52 and the flexible zone 54, although alternate embodiments within the scope of the present invention include transverse absorbent segments having shorter widths or widths as much as 150 millimeters.

The flexible zone 54 occupies the variable width in the absorbent core 50 between the longitudinal absorbent segments 51, 52 and generally aligns under the user's crotch when the absorbent article 10 is properly positioned. The flexible zone 54 generally comprises a minimally absorbent, highly flexible surface having a stepped reduction in thickness and absorbency compared to the absorbent core 50, particularly as compared to the longitudinal absorbent segments 51, 52.

The flexible zone 54 is deformation-assisting. As used herein, the term “deformation-assisting” refers to a zone which is either “flexure-assisting” or “compression-assisting” or a combination of both. As used herein, the term “flexure-assisting” refers to a zone which will support no or minimal bending moment. As used herein, the term “compression-assisting” refers to a zone which will support no or minimal compressive loads.

Various metrics exist to characterize the deformation-assisting property of the flexible zone 54. For example, ASTM 5650-97 (2000) “Standard Test Method for Resistance to Bending of Paper of Low Bending Stiffness” measures the flexure-assisting property. This specific method measures a bending moment of a construction. The lateral compression test described in WO 03/053315 “Absorbent Article with Stabilized Absorbent Structure Having Non-Uniform Lateral Compression Stiffness” provides a method to measure the compression-assisting property. This specific method measures the lateral compression stiffness of a construction.

Qualitatively, the flexible zone 54 generally possesses a lower value for some or all of these measurements when compared to the longitudinal absorbent segments 51, 52. As a result, the flexible zone 54 will preferentially bend, buckle, or compress before and/or to a larger degree than the longitudinal absorbent segments 51, 52 when subjected to a compressive load.

Quantitatively, the measurements may provide a basis for determining, evaluating, and/or varying the effectiveness of the flexible zone 54, depending on the particular application and desired functionality. For example, in particular embodiments, a ratio of a specific measurement, such as the bending moment or maximum lateral compression force, of the flexible zone 54 compared to the same specific measurement of the longitudinal absorbent segments 51, 52 may be less than one. Other particular embodiments may require a ratio that approaches values as low as 0.01.

The width of the flexible zone 54 varies according to the particular embodiment and intended use for the absorbent article 10. In general, the flexible zone 54 has a width of approximately 20-100 millimeters when the longitudinal absorbent segments 51, 52 are spaced apart. Conversely, when the longitudinal absorbent segments 51, 52 are subjected to lateral forces by the user's legs and pushed together, the flexible zone 54 easily folds upon itself, for example in an accordion-like fashion, to occupy minimal width in the absorbent core 50. In a particular embodiment, the absorbent article 10 may be manufactured, packaged, and shipped with the flexible zone 54 in a compressed state such that the flexible zone 54 occupies a minimum width between the longitudinal absorbent segments 51, 52. Prior to use, the wearer removes the absorbent article 10 from the package and rolls, kneads, pulls, stretches, or otherwise manipulates the absorbent article 10 to separate the longitudinal absorbent segments 51, 52 and extend the flexible zone 54 to an uncompressed width.

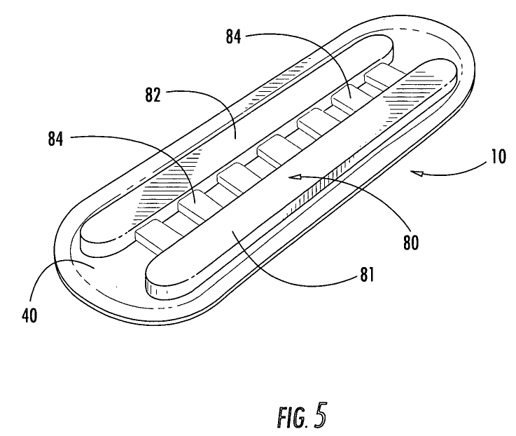

The length of the flexible zone 54 varies according to the particular embodiment and intended use for the absorbent article 10. In general, the flexible zone 54 exists beneath the user's genitalia or crotch area and extends approximately 20-140 millimeters in length along the longitudinal axis X, terminating at or near the transverse absorbent segment 56. The termination of the flexible zone 54 may be rounded, as shown in FIGS. 1 and 2A. Alternately, the flexible zone 54 may terminate in a square shape or taper to a point. In particular embodiments, as shown in FIGS. 3-5, the flexible zone 54 may extend forward and/or rearward along the length of the absorbent article 10.

The flexible zone 54 may also include a surfactant to further minimize absorbency in the flexible zone 54. In alternate embodiments, the flexible zone 54 may include superabsorbent material to provide a thin, lightweight, and flexible absorbent capacity. However, certain structure, additives, or coatings in the flexible zone 54 that could reduce the flexibility, suppleness, or resilience of the flexible zone 54 should be avoided. For example, if an adhesive is used to attach the absorbent core 50 to the cover 20 and/or baffle 40, as previously described, the adhesive should not extend into the flexible zone 54. Similarly, if the garment-facing side of the baffle 40 includes adhesive strips for adhering the absorbent article 10 to the user's undergarment, the adhesive strips should not coincide with the location of the flexible zone 54.

Physically, the flexible zone 54 may simply be an extension of the cover 20, an extension of the baffle 40, a combination of the cover 20 and baffle 40, or it may comprise a thin, separate, minimally absorbent sheet between the cover 20 and baffle 40. One example of a thin, separate, minimally absorbent sheet is a tissue or non-woven web, such as that marketed under the name SONTARA. In particular embodiments, the flexible zone 54 may comprise a collection of free flowing particles. As used herein, the term “free flowing” refers to the ability of the particles to readily move in response to shear forces typically encountered in the use of the absorbent article 10. Examples of free flowing particles include low density foam, polymethylurea (PMU) particles, fiber flakes, and fiber bundles.

The flexible zone 54 combines with the longitudinal absorbent segments 51, 52 in the absorbent core 50 to improve the overall coverage, capacity, visual discretion, and comfort of the absorbent article 10. When the flexible zone 54 is extended, as shown in FIG. 2A, the longitudinal absorbent segments 51, 52 can move with the user's legs to improve comfort, and the combined width of the flexible zone 54 (approximately 20-100 millimeters) and the longitudinal absorbent segments 51, 52 (approximately 10-50 millimeters) covers an effective width of up to 150 millimeters. When the user's legs compress the flexible zone 54, as shown in FIG. 2B, the reduced width of the absorbent core 50 remains comfortable and continues to provide full coverage with little or no deformation or twisting, there by creating a situation for maximum absorbent efficiency.

FIGS. 3 through 5 illustrate alternate embodiments of absorbent cores within the scope of the present invention. The cover and optional transfer layer described in the previous embodiment have been cut away from each figure to better illustrate the absorbent core in each embodiment.

Referring to FIG. 3, an alternate embodiment of an absorbent core 60 again includes two longitudinal absorbent segments 61, 62 separated by a flexible zone 64. In this embodiment, however, the flexible zone 64 simply comprises the previously described baffle 40. In addition, the absorbent core 60 does not include a transverse absorbent segment at either end of the absorbent article 10. Instead, the longitudinal absorbent segments 61, 62 curve inwardly from either end of the absorbent article 10 so that the flexible zone 64 at either end is wider at the ends than at the center. For example, the flexible zone 64 may be approximately 10-20 millimeters wide at the center and over 40 millimeters wide at either end. A further embodiment may reverse the curvature of the longitudinal absorbent segments 61, 62 to provide a flexible zone 64 that is wider at the center than at either end.

Referring to FIG. 4, an absorbent core 70 in this embodiment includes a third rectangle-shaped longitudinal absorbent segment 73 located between two peanut-shaped longitudinal absorbent segments 71, 72. The third absorbent segment 73 is generally a smaller, thinner version of the other two absorbent segments 71, 72 and provides the absorbent article 10 with additional fluid coverage and capacity. In addition, all three longitudinal absorbent segments 71, 72, 73 include transfer splits 78 to increase flexibility and conformity with the user's body. This embodiment may be preferable when the user anticipates extended periods of walking or standing, extended periods between changing absorbent articles, or periods of especially heavy flows of exudates.

Referring to FIG. 5, an absorbent core 80 includes two longitudinal absorbent segments 81, 82 separated by a plurality of transverse flexible strips 84. The transverse flexible strips 84 comprise the same material, provide the identical functions, and occupy the flexible zone described in earlier embodiments. The transverse flexible strips 84 may attach to the longitudinal absorbent segments 81, 82 and/or the cover (not shown) and/or the baffle 40 to enhance structural integrity.

It will be appreciated that the foregoing examples, given for purposes of illustration, are not to be construed as limiting the scope of this invention. Although only a few exemplary embodiments of this invention have been described in detail above, those skilled in the art will readily appreciate that many modifications are possible in the exemplary embodiments without materially departing from the novel teachings and advantages of this invention. Accordingly, all such modifications are intended to be included within the scope of this invention, which is defined in the following claims and all equivalents thereto. Further, it is recognized that many embodiments may be conceived that do not achieve all of the advantages of some embodiments, yet the absence of a particular advantage shall not be construed to necessarily mean that such an embodiment is outside the scope of the present invention.

1. A feminine care absorbent article, comprising:
a. a liquid permeable top sheet on a first side of said feminine care absorbent article;
b. a generally liquid impermeable back sheet on a second side of said feminine care absorbent article;
c. an absorbent core between said top sheet and said back sheet, said absorbent core comprising a first longitudinal segment and a second longitudinal segment transversely separated from said first longitudinal segment, wherein said first and second longitudinal segments have an anterior end and a posterior end; and
d. a flexible zone between said first and second longitudinal segments, said flexible zone having a reduced absorbency compared to said absorbent core.

2. The feminine care absorbent article as in claim 1, wherein each of said first and second longitudinal segments has a transverse width of less than about 20 millimeters.

3. The feminine care absorbent article as in claim 1, wherein said flexible zone has a transverse width of at least 10 millimeters.

4. The feminine care absorbent article as in claim 1, wherein said absorbent core further comprises a transverse segment proximate to said anterior ends of said first and second longitudinal segments.

5. The feminine care absorbent article as in claim 1, wherein said absorbent core further comprises a transverse segment proximate to said posterior ends of said first and second longitudinal segments.

6. The feminine care absorbent article as in claim 1, wherein said flexible zone comprises a superabsorbent.

7. The feminine care absorbent article as in claim 1, wherein said flexible zone comprises a surfactant.

8. The feminine care absorbent article as in claim 1, further including an absorbent strip between said first and second longitudinal segments.

9. The feminine care absorbent article as in claim 1, further including a transverse flexible strip between said first and second longitudinal segments.

10. The feminine care absorbent article as in claim 1, further including a transfer layer between said top sheet and said absorbent core.

11. The feminine care absorbent article as in claim 1, wherein said liquid permeable top sheet peripherally joins to said generally liquid impermeable back sheet.

12. An absorbent article, comprising:
a. a liquid permeable top sheet on a first side of said absorbent article;
b. a generally liquid impermeable back sheet on a second side of said absorbent article;
c. a plurality of longitudinal absorbent segments between said top sheet and said back sheet, said longitudinal absorbent segments having an anterior end and a posterior end; and
d. a flexible zone between said longitudinal absorbent segments, said flexible zone having a reduced thickness compared to said longitudinal absorbent segments.

13. The absorbent article as in claim 12, wherein each of said longitudinal absorbent segments has a width of less than about 20 millimeters.

14. The absorbent article as in claim 12, wherein said flexible zone has a transverse width of at least 10 millimeters.

15. The absorbent article as in claim 12, further including a transverse absorbent segment proximate to said anterior ends of said longitudinal absorbent segments.

16. The absorbent article as in claim 12, further including a transverse absorbent segment proximate to said posterior ends of said longitudinal absorbent segments.

17. The absorbent article as in claim 12, wherein said flexible zone comprises a superabsorbent.

18. The absorbent article as in claim 12, wherein said flexible zone comprises a surfactant.

19. The absorbent article as in claim 12, further including a transverse flexible strip between said longitudinal absorbent segments.

20. The absorbent article as in claim 12, further including a transfer layer between said top sheet and said longitudinal absorbent segments.

21. The absorbent article as in claim 12, wherein said liquid permeable top sheet peripherally joins to said generally liquid impermeable back sheet.

22. A feminine care absorbent article, comprising:
a. a liquid permeable cover on a first side of said feminine care absorbent article;
b. a generally liquid impermeable baffle on a second side of said feminine care absorbent article;
c. an absorbent core between said cover and said baffle having a longitudinal axis and a transverse axis;
d. wherein said absorbent core has a stepped absorbent capacity and a stepped flexibility along said transverse axis.

23. The feminine care absorbent article as in claim 22, wherein said absorbent core has a substantially constant absorbent capacity and a substantially constant flexibility along said longitudinal axis.

24. The feminine care absorbent article as in claim 22, wherein said absorbent core has minimal absorbent capacity and maximum flexibility along said longitudinal axis.

25. The feminine care absorbent article as in claim 22, wherein said liquid permeable cover peripherally joins to said generally liquid impermeable baffle.

26. A feminine care absorbent article, comprising:
a. a liquid permeable cover on a first side of said feminine care absorbent article;
b. a generally liquid impermeable baffle on a second side of said feminine care absorbent article;
c. an absorbent core between said cover and said baffle, said absorbent core comprising a first longitudinal segment and a second longitudinal segment transversely separated from said first longitudinal segment;
d. wherein said absorbent core has an extended position in which said first and second longitudinal segments are transversely separated by approximately 10 millimeters; and
e. wherein said absorbent core has a compressed position in which said first and second longitudinal segments are substantially together.

27. The feminine care absorbent article as in claim 26, wherein said absorbent core has a longitudinal axis having a substantially constant absorbent capacity and a substantially constant flexibility.

28. The feminine care absorbent article as in claim 26, wherein said absorbent core has minimal absorbent capacity and maximum flexibility along said longitudinal axis.

29. The feminine care absorbent article as in claim 26, wherein said liquid permeable cover peripherally joins to said generally liquid impermeable baffle.
